SEQUENCE LISTING (13 sequences)

SEQ ID NO: 1 - protein, 13 aa, artificial sequence
  synthetic peptide
RAAAEEGHIIPRS

SEQ ID NO: 2 - protein, 13 aa, artificial sequence
  synthetic peptide
KSQEIIPFPSTKM

SEQ ID NO: 3 - protein, 15 aa, artificial sequence
  synthetic peptide
KLKSQEIIPFPSTKM

SEQ ID NO: 4 - protein, 19 aa, artificial sequence
  synthetic peptide
KTYVDLTNEETTDSTTSKI

SEQ ID NO: 5 - protein, 19 aa, artificial sequence
  synthetic peptide
RALNSYFEPPVEESALERR

SEQ ID NO: 6 - protein, 21 aa, artificial sequence
  synthetic peptide
KMQEAPESATVIFAGDTNLRD

SEQ ID NO: 7 - protein, 24 aa, artificial sequence
  synthetic peptide
RSSNYEIITGHEEGYFTAIMLKKS

SEQ ID NO: 8 - protein, 11 aa, artificial sequence
  synthetic peptide
RSLDLLGLEKL

SEQ ID NO: 9 - protein, 11 aa, artificial sequence
  synthetic peptide
RRPETISEPKT

SEQ ID NO: 10 - protein, 13 aa, artificial sequence
  synthetic peptide
RAAAEEGHIIPRS

SEQ ID NO: 11 - protein, 15 aa, artificial sequence
  synthetic peptide
KLKSQEIIPFPSTKM

SEQ ID NO: 12 - protein, 19 aa, artificial sequence
  synthetic peptide
RALNSYFEPPVEESALERR

SEQ ID NO: 13 - protein, 23 aa, artificial sequence
  synthetic peptide
RSSNYEIITGHEEGYFTAIMLKK

(19) United States
(12) Patent Application Publication
(10) Pub. No.: US 2013/0345162 A1
(43) Pub. Date: Dec. 26, 2013

(54) NOVEL ANTI-VIRAL THERAPEUTIC

(71) Applicant: The Regents of the University of California, Oakland, CA (US)

(72) Inventors: Bert L. Semler, San Clemente, CA (US); Richard Virgen-Slane, Irvine, CA (US); Janet M. Rozovics, Oakland, CA (US); Paul D. Gershon, Oakland, CA (US)

(73) Assignee: The Regents of the University of California, Oakland, CA (US)

(21) Appl. No.: 13/867,818

(22) Filed: Apr. 22, 2013

Related U.S. Application Data
(60) Provisional application No. 61/636,253, filed on Apr. 20, 2012.

(51) Int. Cl. C07H 19/20; C12N 9/22; C12Q 1/70

(52) U.S. Cl. CPC C07H 19/20; C12Q 1/70; C12N 9/22
USPC 514/46; 435/5; 435/199

(57) ABSTRACT

Anti-viral therapeutics for use in viral targets having VPg unlinkase activity, including the broad class of picornaviruses, and therapeutic methods directed at suppression of such activity are provided. Such anti-viral therapies for use against human rhinovirus, for example, are particularly desirable as they would lessen both the severity and duration of upper respiratory distress in both normal and asthmatic individuals. Assays and purification protocols for detecting and purifying VPg unlinkase are also provided.

### CROSS-REFERENCE TO RELATED APPLICATIONS

This application claims priority to Provisional Application No. 61/636,253 filed Apr. 20, 2012, the disclosure of which is incorporated herein by reference.

### STATEMENT OF FEDERAL SUPPORT

This invention was made with government support under Public Health Service Grants AI 26765 and AI 07319 issued by the National Institutes of Health (NIH).

### FIELD OF THE INVENTION

The present invention is directed to novel anti-viral therapeutics, and more particularly to therapeutics involving the suppression of VPg unlinkase activity.

### BACKGROUND OF THE INVENTION

Dating back to the 1970's, it has been well documented that viral infections of the respiratory tract can precipitate asthma attacks and increase the severity of the resulting sequelae. (See, e.g., Lambert, H. P. and H. Stern, Br. Med. J. 3:323-327 (1972); and Minor, T. E., et al., Am. Rev. Respir. Dis. 113:149-153 (1976), the disclosures of which are incorporated herein by reference.) The increase in severity of symptoms is especially problematic in children, for whom it has been estimated that viral infections are associated with 80-85% of asthma exacerbations, including severe wheezing. (See, e.g., Johnston, S. L., et al., BMJ 310:1225-1229 (1995); Minor, T. E., et al., J. Pediatr. 85:472-477 (1974); and Minor, T. E., et al., JAMA 227:292-298 (1974), the disclosures of which are incorporated herein by reference.) A recent study using RT-PCR analysis of respiratory secretions from a cohort of children reported that rhinovirus was the most prevalent respiratory virus in patients with asthma exacerbations. (Khetsuriani, N., et al., J. Allergy Clin. Immunol. 119:314-321 (2007), the disclosure of which is incorporated herein by reference.) Experimental rhinovirus infections of human volunteers have been used to document an increase in the concentration of pro-inflammatory cytokines in nasal secretions, resulting in increased local inflammation and activation of macrophages to further exacerbate inflammatory processes in the airways [for review, see Busse, W. W., J. Clin. Pharmacol. 39:241-245 (1999); and Gem, J. E. and W. W. Busse, Clin. Microbiol. Rev. 12:9-18 (1999), the disclosures of which are incorporated herein by reference]. In addition, cytokines such as IFN-γ and TNF-α that are produced as a result of macrophage activation can stimulate the increased expression of adhesion proteins like ICAM-1 by epithelial cells. (See, Look, D. C., et al., Am. J. Physiol 263:L79-L87 (1992); and Tosi, M. F., et al., Am. J. Respir. Cell Mol. Biol. 7:214-221 (1992), the disclosures of which are incorporated herein by reference.) Given that ICAM-1 is the cellular receptor for the majority of human rhinoviruses, this up-regulation increases the number of potential binding sites for infectious virions as well as increasing the number of ligands for eosinophils and neutrophils in the bronchial epithelium, since these inflammatory cells bind via ICAM-1 as well. (See, Greve, J. M., et al., Cell 56:839-847 (1989); and Staunton, D. E., et al., Cell 56:849-853 (1989), the disclosures of which are incorporated herein by reference.) Finally, based upon the results from experimental infections of volunteers, it appears that rhinovirus infections up-regulate factors involved in allergic inflammation, a process that alters airway functions and leads to wheezing. (See, Busse, W. W. cited above.) These observations provide a significant link between rhinovirus infections and exacerbations of asthma sequelae, providing a strong rationale for the development of anti-viral therapies against rhinoviruses. (See, Greenberg, S. B., Semin. Respir. Grit Care Med. 28:182-192 (2007), the disclosure of which is incorporated herein by reference.) Despite these links and the continued interest in finding therapeutics against these diseases, no effective therapy for these viruses has yet been developed.

Picornaviruses like poliovirus, human rhinovirus, and foot-and-mouth disease virus utilize a small viral protein (VPg) as a primer for viral RNA synthesis, which results in the linkage of all nascent viral RNAs to VPg via an 04-(5′-uridylyl)tyrosine bond. (See, Rozovics, J. M. and Semler, B. L. Genome replication I: the players. in The Picornaviruses, edited by E. Ehrenfeld, E. Domingo, and R. P. Roos (ASM Press, Washington, D.C., 2010), pp. 107-125, the disclosure of which is incorporated herein by reference.) Following genome release from the infecting virion, however, the VPg-RNA linkage of virion RNA (vRNA) is short-lived. Upon polysome association, VPg is removed from vRNA by a cellular enzyme, which will be referred to as “VPg unlinkase”. The critical role this enzyme plays in the replication cycle of many viruses makes it an excellent target for therapeutics. Moreover, since its discovery in 1978, several studies have described the partial purification and biochemical characterization of VPg unlinkase and explored its role(s) during picornavirus infection. (See, Ambros, V., Pettersson, R., and Baltimore, D., Cell 15, 1439-1446 (1978)I; Ambros, V., and Baltimore, D., J. Biol. Chem. 255, 6739-6744 (1980); Yusupova, R. A., Gulevich, A. Y., and Drygin, Y. F. Biochemistry Mosc. 65, 1219-1226 (2000); Drygin, Y. Nucleic Acids Res. 26, 4791-4796 (1998); Sangar, D. V. et al. J. Virol. 39, 67-74 (1981); Rozovics, J. M., Virgen-Slane, R., and Semler, PLoS ONE 6, e16559 (2011); Gulevich, A. Y., Yusupova, R. A., and Drygin, Y. F., Biochemistry Mosc 67, 615-621 (2002); and Nomoto, A., et al., Proc. Natl. Acad. Sci. USA 74, 5345-5349 (1977), the disclosures of which are incorporated herein by reference.) Despite these efforts, the cellular identity of VPg unlinkase remains elusive, as have therapeutics based on this promising target.

### BRIEF SUMMARY OF THE INVENTION

The current invention is directed generally to therapeutics based on VPg unlinkase suppressors.

In one embodiment, the invention is directed to anti-viral therapeutics for viruses requiring VPg unlinkase activity, including a therapeutically effective amount of a VPg unlinkase enzyme suppressor.

In another embodiment, the invention is directed to a method of treating a viral infection for a virus requiring VPg unlinkase activity including administering a therapeutically effective amount of a VPg unlinkase enzyme suppressor.

In still another embodiment, the invention is directed to a method of screening for VPg suppressing viral therapeutics including:
obtaining a plurality of potential anti-viral compounds; and
determining which of said anti-viral compounds have VPg unlinkase enzyme suppressing activity.

In some such embodiments, the VPg unlinkase enzyme is TDP2.

In other such embodiments, the suppressor is 5′FSBA.

In still other such embodiments, the virus is selected from the group consisting of picornaviruses, plant viruses and caliciviruses. In one such embodiment, the picornavirus is selected from the group consisting of CVB3, EMCV, HRV16 and PVI. In another such embodiment, the plant virus is selected from the group consisting of cowpea mosaic virus, sobemovirus, and rye grass mottle. In still another such embodiment, the calicivirus is selected from the group consisting of norwalk, feline calicivirus, and murine norovirus.

In yet another embodiment, the invention is directed to an assay for monitoring the release of VPg from genomic RNA generally including:
isolating a virion RNA containing an ³⁵S-labeled VPg;
producing a ³⁵S-VPg-nonanucleotide substrate;
entering the ³⁵S-VPg-nonanucleotide into a Tris-Tricine polyacrylamide gel; and
monitoring the cleavage of the VPg from the substrate.

In some such embodiments, the step of isolating includes:
labeling a virus-infected cell with a [³⁵S]methionine marker; and
purifying the labeled virion RNA from the virus-infected cells.

In other such embodiments the purifying comprises sucrose gradient fractionation.

In still other such embodiments the producing comprises digesting the labeled VPg with RNAse T1.

In still another embodiment, the invention is directed to a VPg unlinkase purification protocol including:
providing a VPg unlinkase containing target cell homogenate;
producing a supernatant form said target cell homogenate; and
sequentially fractionating the supernatant by heparin Sepharose, ssDNA-cellulose, anion exchange, size exclusion, and cation exchange chromatography to produce a substantially homogeneous enzyme preparation.

In some such embodiments the supernatant is produced by subjecting the target cell homogenate to high-speed centrifugation.

### BRIEF DESCRIPTION OF THE DRAWINGS

Various examples of the present invention will be discussed with reference to the appended figures and data results, wherein:

FIG. 1 provides a schematic of an exemplary picornavirus replication cycle and VPg unlinkase activity, where VPg (orb at 5′-terminus of the vRNA is shown), and VPg unlinkase is depicted as the “pac man” figure, ribosomes are depicted as (filled ovals), in the bottom panel, (+) strand RNA is indicated by a long, white rectangle with a 3′ poly(A) tract, while (−) strand RNA is indicated by a long black rectangle with a 5′ poly(U) tract.

FIG. 2 provides an image of an assay for unlinkase activity following anion exchange chromatography.

FIG. 3 provides data showing that single-strand DNA (ssDNA) is 100-fold more efficient at inhibiting VPg unlinkase activity than synthetic RNA with or without a 5′-tyrosyl-RNA bond.

FIG. 4 provides data from isolation of VPg unlinkase from HeLa cell homogenate, wherein: (a) provides a table summarizing the purification of VPg unlinkase is shown (activity units were quantified from the relative levels of VPg signal generated in a 20 μ1 reaction incubated for 3 minutes at 30° C. (protein concentrations were determined by Bradford assay and SDS-PAGE analysis), (b) shows a SDS-PAGE analysis (protein gel stained with SYPRO Ruby) of the purification process (labeled by purification step, see FIG. 2a) shows the isolation of p38, where a longer exposure of the gel (right) was required to visualize proteins in lanes E and F, and (c) an analysis of purification step F, shows the co-elution of VPg unlinkase activity (top) with p38 (bottom).

FIG. 5 provides data showing the detection of VPg unlinkase.

FIG. 6 provides data from the identification of p38 as TDP2, where: (a) shows a mass spectrometry analysis of p38 isolated from lanes F12 (p38-F12, top table) and F13 (p38-F13, bottom table) of the polyacrylamide gel shown in FIG. 3c (bottom panel) identified several tryptic peptides corresponding to TDP2, (b) shows a western blot analysis using anti-TDP2 polyclonal antibody confirms the isolation of TDP2 by our purification protocol, and (c) shows the relative TDP2 expression levels in different cellular extracts correlate with the VPg unlinkase activity detected p7:reviously HeLa (human cervical carcinoma cell line)>K562 (human myeloid leukemia cell line)>NGP (human neuroblastoma cell line)>SKOV3 (human ovarian carcinoma cell line)>RRL (rabbit reticulocyte lysate).

FIG. 7 provides data showing recombinant GST-TDP2 has authentic unlinkase activity, where: (a) shows equivalent amounts of partially purified VPg unlinkase and GST-TDP2 both unlinked VPg from [³⁵S]VPg-PV RNA (top), without any apparent degradation of PV1 RNA (bottom), (b) shows that increasing amounts of partially purified VPg unlinkase and GST-TDP2, but not GST, unlinked VPg from [³⁵S]VPg-PV RNA and [³⁵S]VPg HRV-PV RNA (reactions containing RNase A were included to generate markers for VPg-pUp (lanes 6 and 13)).

FIG. 8 provides data showing all three forms of TDP2 co-eluted with the corresponding species of VPg unlinkase activity detected in crude extract (a); and the detection of at least three forms of TDP2 with apparent molecular masses ranging from 26 to 50 kDa (b).

FIG. 9 provides a schematic of the molecular structure of 5′FSBA.

FIG. 10 provides an image of an assay for unlinkase activity after 5′FBSA inhibition.

FIG. 11 provides a data graph showing 5′FSBA inhibition of unlinkase.

FIG. 12 provides a data graph showing poliovirus vRNA transfection time course with and without TDP2 suppression.

FIG. 13 provides a data graph showing CVB3 single cycle growth curves with and without TDP2 suppression.

### DETAILED DISCLOSURE OF THE INVENTION

Turning now to the diagrams and figures, therapeutics, including anti-viral therapeutics, for use in viral targets having VPg-RNA covalent linkages, including the broad class of picornaviruses are described. Such anti-viral therapies for use against human rhinovirus, for example, are particularly desirable as they would lessen both the severity and duration of upper respiratory distress in both normal and asthmatic individuals. However, to date the available anti-viral treatments targeting specific steps in the human rhinovirus replication cycle have not been shown to be efficacious. The reason for this repeated failure is that most of these treatments require a viral-specific target, such as a unique enzyme in an infected cell like a viral proteinase. However, these proteinases are structurally similar to host proteases. As a result, compounds targeting those viral proteinases typically also have toxicity to the host cells. Another problem is that the viral RNA-dependent RNA polymerase typically generates a great number of replication errors. Most of these errors lead to non-viable viruses, but with some frequency, reversions or second site suppressor mutations occur that produce viable virus variants and confer resistance to the treatment. Accordingly, in some embodiments anti-viral therapeutics that suppress the VPg unlinkase interface between the target virus and host are described.

Specifically, in some embodiments therapeutics are described that target the cellular activity (termed “unlinkase”) that removes a small viral peptide (VPg) from the 5′ end of genomic RNAs from many viruses (including human rhinovirus) by cleaving a protein-nucleotide bond prior to the onset of viral protein synthesis. Data provided in support of the therapeutic efficacy of this approach demonstrates the identity of at least one protein(s) responsible for this unlinkase activity, and the consequences for viral replication when the activity of the protein(s) is down-regulated during a rhinovirus infection of human cells. More specifically, in many embodiments therapeutics directed at suppressing the 5′-tyrosyl-RNA phosphodiesterase activity of the DNA repair enzyme, 5′ tyrosyl-DNA phosphodiesterase-2 (TDP2) are described.

It has been determined that targeting TDP2 has two advantages, first the removal of VPg from RNA is a common feature of virus replication, and is a universal feature of the replication of the important picornavirus family, which includes, for example, rhinovirus, poliovirus, enterovirus 71, coxsackievirus, etc. Second, TDP2 is expressed from a cellular gene, which means that any resistance to drugs targeting this enzyme would arise from the virus evolving a novel mechanism for regulating viral replication, a much more unlikely circumstance. To understand why this is true, and why the therapeutics embodied herein are particularly promising, it is necessary to understand the mechanism of viral replication and the function of TDP2.

### Overview of Viral Mechanism

Human rhinovirus (HRV) is a member of the Picornaviridae, positive-strand, cytoplasmic RNA viruses that cause a significant number of diseases in humans such as the common cold, poliomyelitis, myocarditis, and hepatitis. Although the diseases caused by picornaviruses are diverse, the genome organization and mechanisms of gene expression are highly conserved among family members. As a result, studying picornavirus gene expression has provided critical information into the mechanisms by which picornaviruses cause disease and has provided insights into eukaryotic gene expression and the cellular proteins involved in this process. For example, using the picornavirus as an example, it is possible to outline the mechanisms of HRV gene expression via cap-independent translation initiation and viral RNA replication as they relate to the therapeutics outlined in the application.

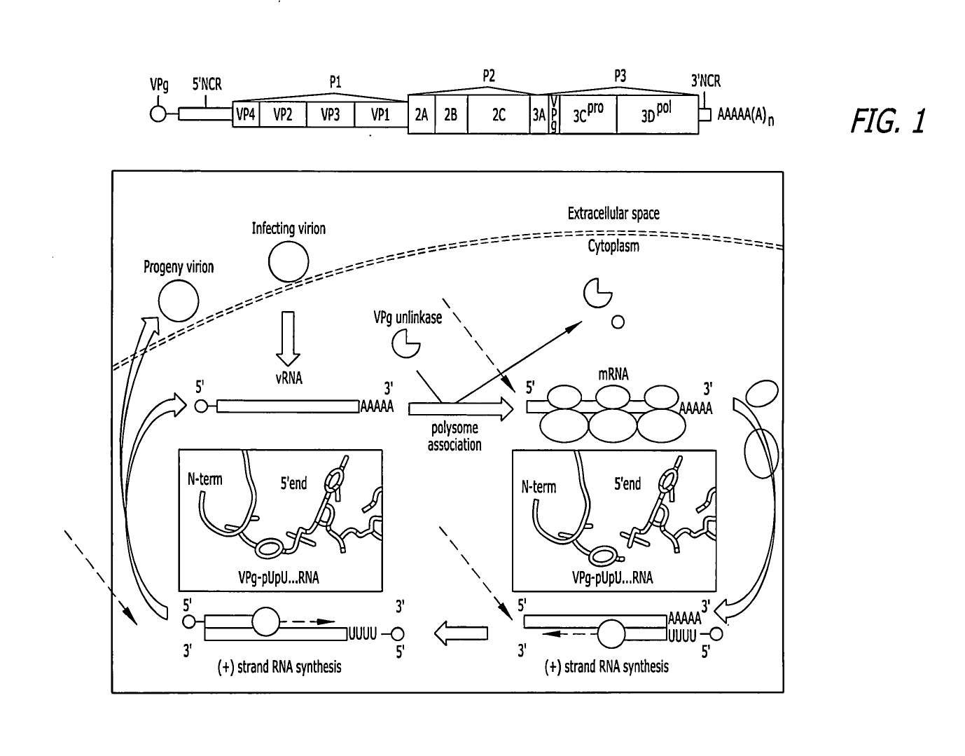

Of specific relevance to the instant invention, HRV contains a 7.2 kb positive-sense, single-stranded RNA genome. Instead of a 7-methyl guanosine cap structure linked to the 5′ end of the genomic RNA, it has a small, viral protein called VPg (see FIG. 1). The genomic RNA of HRV (like that of all picornaviruses, and many other viruses, including, for example, many plant viruses and caliciviruses) is identical to viral mRNA, except that viral mRNA associated with polysomes in infected cells lacks VPg. (See e.g., Nomoto, A., et al., Proc. Natl. Acad. Sci. U.S.A 74:5345-5349 (1977), the disclosure of which is incorporated herein by reference.) As will be discussed further (below), VPg is cleaved from the viral RNA early during infection by a cellular enzyme, since no enzymatically active viral proteins enter the cell with the viral RNA. (See, Ambros, V. and D. Baltimore. (1978), cited above.)

Genomic RNA has a long (˜600 nt.), highly-structured 5′ noncoding region (5′ NCR) that contains the internal ribosome entry site (IRES) necessary for translation initiation. The coding region of the viral genome specifies the structural and non-structural viral proteins, divided into three primary precursor molecules (P1, P2, and P3). The structural proteins that comprise the viral capsid are derived from the P1 portion of the polyprotein, and the non-structural proteins are found in the P2 and P3 regions. The genome organization for human rhinovirus is provided schematically in FIG. 1 (top). The P2 region of the genome encodes proteins that are important for membrane rearrangement in infected cells and required for HRV RNA replication. Additional viral proteins that are crucial for replication, such as VPg, the viral proteinase 3C/3CD and the viral RNA-dependent RNA polymerase 3D^{pol}, are encoded within the P3 region of the genome. HRV genomic RNAs also contain a 3′ NCR (˜50 nucleotides) and a 3′ poly(A) tract, which are thought to be involved in RNA replication and translation. The 3′NCR may have a role in increasing the efficiency of viral RNA replication and in tissue tropism. (See, Brown, D. M., et al., J. Virol. 78:1344-1351 (2004), the disclosure of which is incorporated herein by reference.) The 3′ poly(A) tract is essential for virus infectivity and is thought to increase the stability of viral RNA. (See, Spector, D. H. and D. Baltimore, Proc. Natl. Acad. Sci. U.S.A 71:2983-2987 (1974), the disclosure of which is incorporated herein by reference.) Although not to be bound by theory, there is also evidence that interactions between the 5′ and 3′ ends of viral RNA may occur through binding of viral and host cell factors, perhaps mediating initiation of negative strand RNA synthesis. (See, Herold, J. and R. Andino, Mol. Cell. 7:581-591 (2001), the disclosure of which is incorporated herein by reference.)

RNA sequences and structures within the 5′ NCR of HRV genomic RNA mediate translation of a single polyprotein of approximately ˜250 kDa, via a mechanism of cap-independent translation known as internal ribosome entry. (See, Jang, S. K., et al., Virol. 62:2636-2643 (1988); and Pelletier, J. and N. Sonenberg. Nature 334:320-325 (1988), the disclosures of each of which are incorporated herein by reference.) The product of translation is a large polyprotein that is processed by the viral proteinases 2A and 3C/3CD to generate mature structural and non-structural viral protein products. Due to the limited coding capacity of picornavirus genomes, precursor polyproteins and mature cleavage products actively participate in viral processes. An additional consequence of having a very small genome is that HRV (like all picornaviruses) must utilize host cell functions in several aspects of its intracellular replication cycle, including translation, membranous vesicle formation, and RNA synthesis. A number of host proteins have been identified that interact with either viral RNAs or viral proteins, and their precise functions in the picornavirus life cycle are the subjects of ongoing research studies. (See, Ahlquist, P., et al., J. Virol. 77:8181-8186 (2003); and Bedard, K. M. and B. L. Semler, Microbes and Infection 6:702-713 (2004), the disclosures of each of which are incorporated herein by reference.)

As noted above, and shown schematically in FIG. 1 (bottom), an important step in HRV replication (and that of all picornaviruses) that requires a host function(s) is the removal of VPg from the 5′ end of genomic RNA that produces viral mRNA found on translating ribosomes. Following translation, the viral mRNA is used as a template for (−) strand RNA synthesis by the viral polymerase ((3D^{pol}) represented by ovalsin FIG. 1). The (−) strand RNA is then used by the viral polymerase (3D^{pol} in the example shown in FIG. 1) as a template for (+) strand RNA synthesis to generate progeny RNAs, which are either packaged or unlinked and translated.

This activity has received relatively little attention beyond the initial studies describing the nature of the linkage between picornavirus RNAs and the genome-linked protein VPg and the subsequent studies describing an activity found in both the nucleus and cytoplasm of uninfected cells (later termed “unlinkase”) that cleaves this bond. (See, Ambros, V. and D. Baltimore, J. Biol. Chem. 253:5263-5266 (1978); Ambros, V. and D. Baltimore, 255:6739-6744 (1980); and Ambros, V., R. F. Pettersson, and D. Baltimore, Cell 15:1439-1446 (1978), the disclosures of each of which are incorporated herein by reference.) The 5′ uridylyl-tyrosine bond between HRV virion RNA and VPg is shown in FIG. 1 (bottom). The 5′ tyrosyl-RNA phosphodiester chemical structure (based on uridylylated foot-and-mouth disease virus VPg; PDB 2F8E is shown before (bottom panel, left box) and after hydrolysis (bottom panel, right box).

The initial characterization of unlinkase activity showed that it requires Mg²⁺ for activity, is sensitive to heat, SDS, Zn²⁺, and EDTA, and is resistant to translation inhibitors, RNAse, and protease inhibitors. (See, Rothberg, P. G., et al., Proc. Natl. Acad. Sci. U.S.A 75:4868-4872 (1978); and Sangar, D. V., et al., J. Virol. 39:67-74 (1981), the disclosures of each of which are incorporated herein by reference.) In addition, unlinkase will not cleave a synthetically-generated 5′ tyrosyl-DNA bond. (See, Shabanov, A. A., et al., Biokhimiia. 61:1106-1118 (1996), the disclosure of which is incorporated herein by reference.) However, attempts to purify and identify the protein have been unsuccessful due to the lack of a suitable assay for activity and to the possible dissociation of a multi-subunit protein complex during one or more of the steps in purification.

### Characterization of VPg Unlinkase

As mentioned above, previous research efforts have attempted to discover the identity of VPg unlinkase activity. However, these efforts were unsuccessful due to the lack of a suitable assay for the release of VPg from genomic RNA, and due to the inability to identify proteins from complex mixtures via mass spectrometry and protein sequence analysis. Specifically, VPg encoded in either the HRV or poliovirus genome contains no methionine or cysteine residues, thereby precluding conventional labeling with, for example, ³⁵S. Because VPg is very small (either 22 or 23 amino acids, depending on the picornavirus), it readily diffuses from polyacrylamide gels during the buffer rinses required for membrane transfer, thereby eliminating Western blot analysis for detecting VPg following incubations with sources of unlinkase activity (e.g., extracts from uninfected, cultured human cells). Accordingly, many embodiments of the invention are directed to a facile assay for unlinkase activity as well as methods for protein purification.

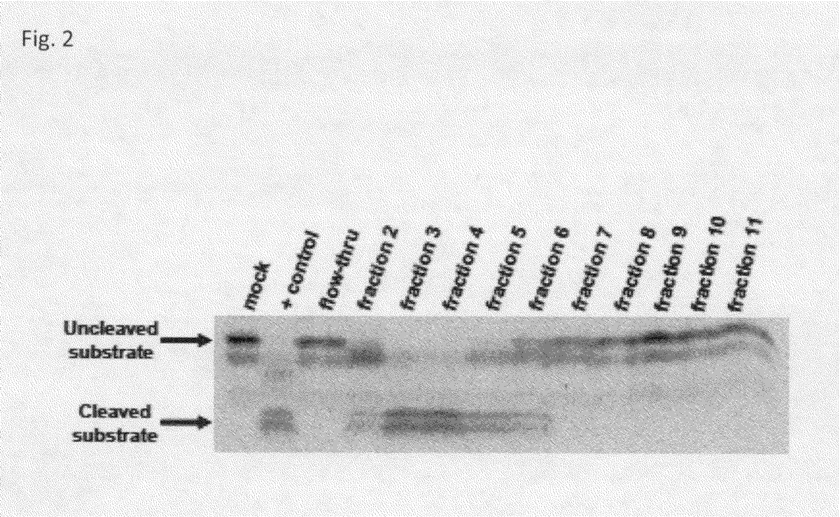

In establishing an assay for unlinkase activity a genetically engineered poliovirus mutant that encodes two methionine residues (wild type poliovirus and HRV VPg proteins that do not contain methionine) was used. (See, Kuhn, R. J., et al., Proc. Natl. Acad. Sci. U.S.A. 85:519-523 (1988), the disclosure of which is incorporated herein by reference.) By labeling poliovirus-infected HeLa cells with [³⁵S]methionine and purifying virus particles using sucrose gradient fractionation, it was possible to isolate virion RNA containing ³⁵S-labeled VPg. Because there is a G residue encoded nine nucleotides from the 5′ end of viral RNA, labeled virion RNA was digested with RNAse T1 to generate a ³⁵S-VPg-nonanucleotide substrate that could readily enter the Tris-Tricine, high percentage polyacrylamide gels required to resolve VPg from the uncleaved substrate. Ambros and Baltimore had previously shown that these nine nucleotides attached to VPg were the minimum number required for substrate cleavage by unlinkase. (See, Ambros, V. and D. Baltimore (1980), cited above.) As shown in FIG. 2, the above-noted features generate a reliable assay for unlinkase activity.

Accordingly, some embodiments are directed to an assay for the release of VPg from genomic RNA generally comprising:
Isolating virion RNA containing ³⁵S-labeled VPg by labeling virus-infected cells with [³⁵S]methionine and purifying the labeled, virus-infected cells;
Producing a ³⁵S-VPg-nonanucleotide substrate by digesting the labeled VPg with RNAse T1;
Entering the ³⁵S-VPg-nonanucleotide into a Tris-Tricine polyacrylamide gel; and
Monitoring the cleavage of the VPg from the substrate.

Although one potential assay is described above, it should be understood that other methods of generating suitably labeled VPg substrates may be devised based on the general guidelines provided above. For example, although in the above discussion poliovirus infected HeLa calls were used, an assay could be based on any viral infected cell demonstrating VPg unlinkase activity. Likewise, although the above technique uses sucrose gradient fractionation to isolate virion RNA, any suitable fractionation technique may be used with the assay. Similarly, although a specific RNAse T1 digestion and Tris-Tricine SDS-PAGE techniques are described, suitable replacements may be found that do not alter the efficacy of the described assay.

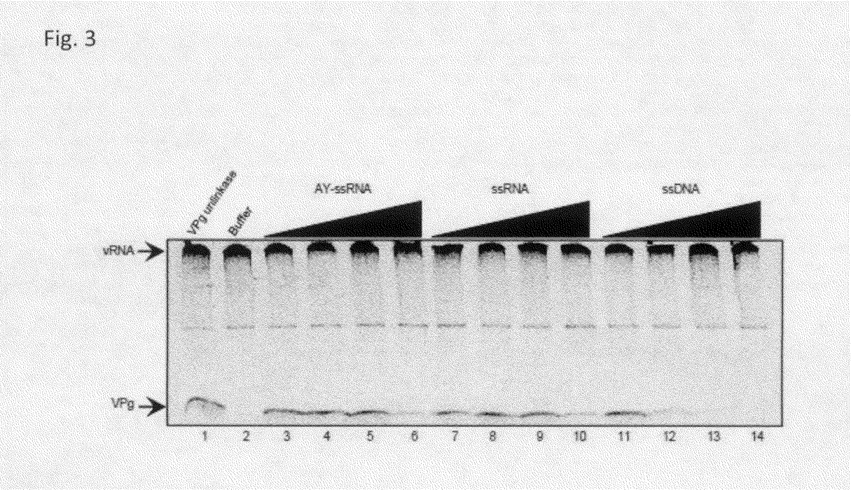

To identify VPg unlinkase, a novel purification procedure was also developed using the above-described VPg unlinkase activity assay, which resolves ³⁵S-methionine radiolabeled VPg released from poliovirus vRNA ³⁵S(-PVI-RNA) by Tris-tricine SDS-PAGE analysis. (See, Rozovics, J. M., Virgen-Slane, R., and Semler, B. L., (2011), cited above.) The assay was optimized for rapid detection of VPg unlinkase activity (see Exemplary Embodiments, below). During the development of this purification scheme, different synthetic compounds and nucleic acids were also screened as competitive inhibitors to be used in the affinity purification of VPg unlinkase. It was observed that a single-strand DNA (ssDNA) is 100-fold more efficient at inhibiting VPg unlinkase activity than synthetic RNA with or without a 5′-tyrosyl-RNA bond (FIG. 3). This key finding prompted the inclusion of ssDNA-cellulose in the purification protocol.

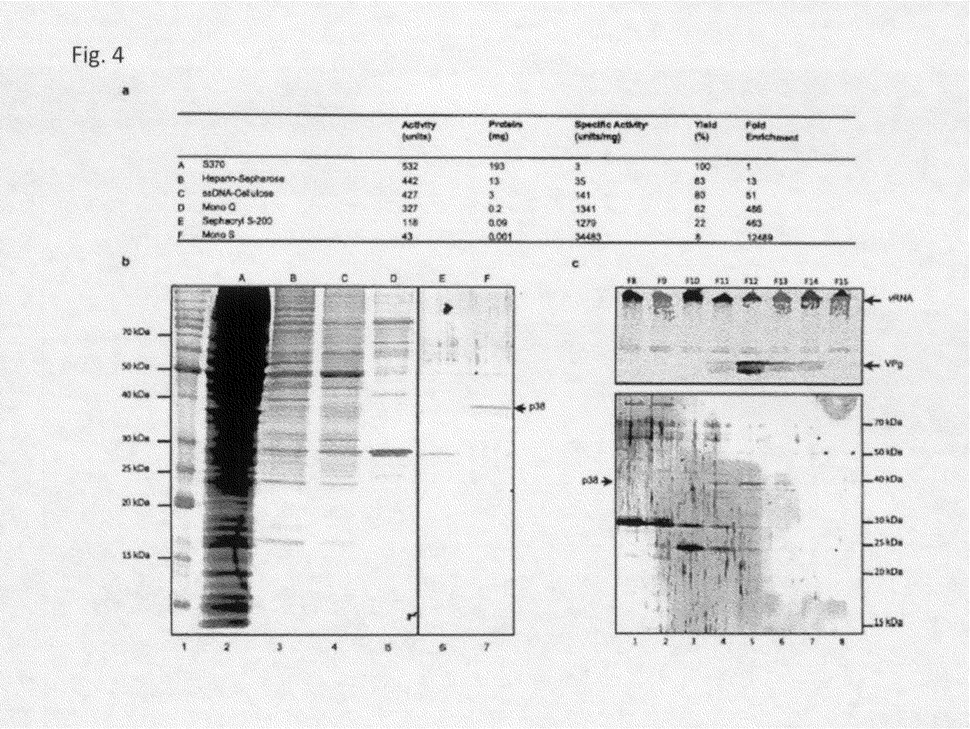

Because VPg unlinkase activity is found in both cytoplasmic and nuclear extracts the purification procedure was initiated using total cell homogenate from uninfected HeLa cells. (See, Ambros, V., et al., Cell (1978), cited above.) After subjecting the homogenate to high-speed centrifugation to pellet cellular debris and large complexes containing nucleic acid-binding proteins, the supernatant (S370), which contained −75% of the initial activity (data not shown), was fractionated sequentially by heparin Sepharose, ssDNA-cellulose, anion exchange, size exclusion, and cation exchange chromatography, resulting in the generation of a nearly homogeneous enzyme preparation in which activity was enriched by >10.000-fold (FIG. 4a).

Accordingly, in many embodiments a VPg unlinkase purification protocol includes:
Subjecting a target cell homogenate to high-speed centrifugation to pellet cellular debris and large complexes containing nucleic acid-binding proteins to form a supernatant;
Sequentially fractionating the supernatant by heparin Sepharose, ssDNA-cellulose, anion exchange, size exclusion, and cation exchange chromatography; and
Thereby generating a nearly homogeneous enzyme preparation in which activity was greatly enriched (in some instance by >10.000-fold).

Although a specific purification protocol for VPg unlinkase is described above, it should be understood that minor modifications to the process may be made, such as, for example, the inclusion of additional fractionation methods or various centrifugation protocols that do not impact the overall efficacy of the technique. For example, although the purification protocol was performed in this example on HeLa cell homogenate it will be understood that any cell homogenate having VPg unlinkase activity may be purified in a similar manner.

Analysis of Polypeptide (p38) as TDP2

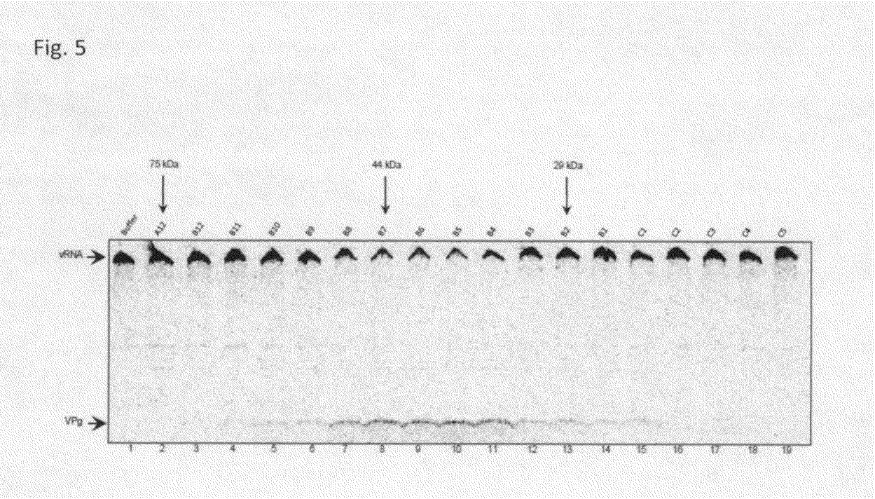

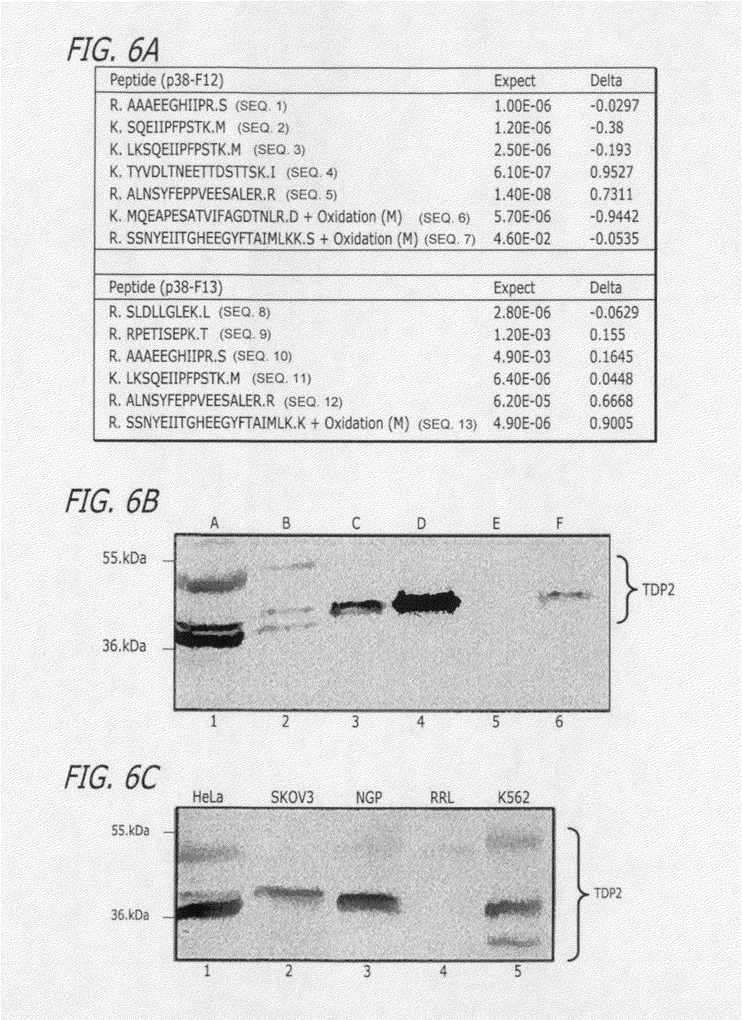

The resulting 38 kDa polypeptide (p38) isolated by this purification scheme (FIG. 4b, lane F) corresponded in size with the VPg unlinkase detected during the initial characterization of this activity (FIG. 5). Analysis of fractions from purification step F (cation exchange chromatography) verified the coelution of p38 (FIG. 4c, bottom panel) with VPg unlinkase activity (FIG. 4c, top panel). The protein corresponding to p38 was excised from two different lanes of a polyacrylamide gel (FIG. 4c, lanes 5 and 6, bottom panel) and subjected to trypsin digestion followed by nanoLC-MS/MS analysis, unequivocally identifying p38 as TDP2 (FIG. 6a), a Mg²⁺/Mn²⁺ dependent cellular hydrolase known to cleave the 5′-tyrosyl-DNA bond generated as a result of topoisomerase-mediated DNA damage. (See, Cortes Ledesma, F. et al., Nature 461, 674-678 (2009), the disclosure of which is incorporated herein by reference.) This protein is also known as TTRAP and EAPII, and has been shown to function in transcriptional regulation, signal transduction, and protein-protein interactions linked to possible roles in neuronal development and cancer progression. (See, Li, C., et al., Cell Cycle 10, 3274-3283 (2011), the disclosure of which is incorporated herein by reference.) In addition, TDP2 is found in both the nucleus and the cytoplasm of mammalian cells in agreement with the original report of VPg unlinkase activity by Ambros and Baltimore. (Ambros, V., et al., (1978), cited above.) The presence of TDP2 in the purified material was verified by Western blot analysis using a commercially available polyclonal antibody (FIG. 6b).

To assess the correlation of TDP2 and VPg unlinkase activity, the correlation of TDP2 expression levels in different cells with their VPg unlinkase activity was determined. The observed expression profile of TDP2 by Western blot analysis (FIG. 6c) was consistent with the relative abundance of VPg unlinkase activity reported previously (Rozovics, et al., (2011), cited above) for extracts from the following cell lines (from highest to lowest activity): HeLa>K562>NGP>SKOV3>RRL. These observations allow for the conclusion that TDP2 expression levels correlate with levels of VPg unlinkase activity.

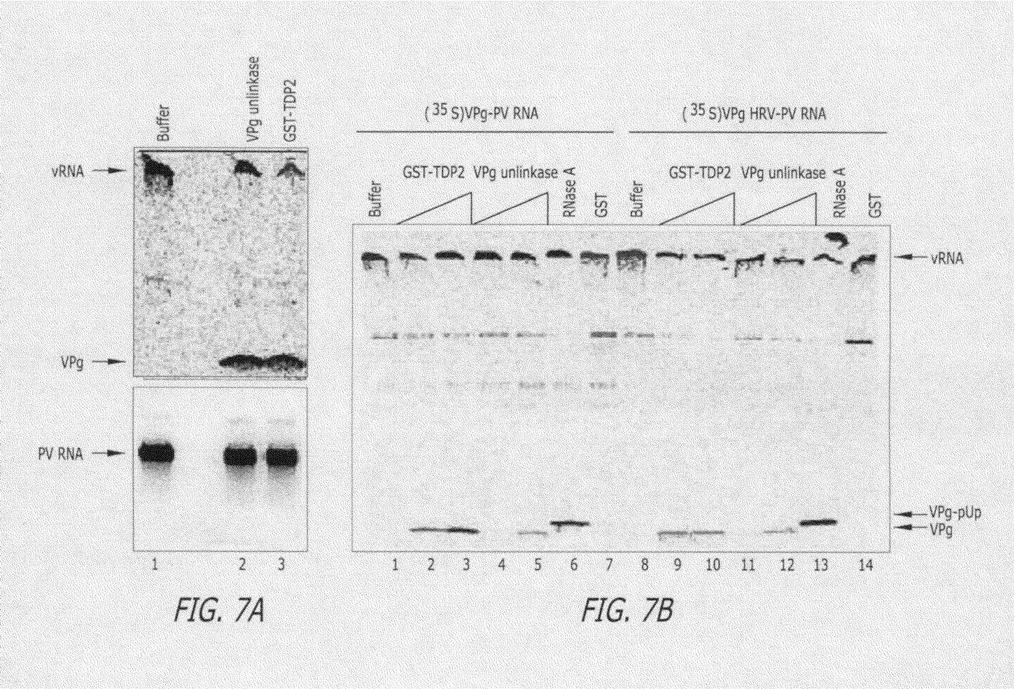

To confirm that TDP2 is VPg unlinkase, recombinant GST-tagged TDP2 was purified from E. coli and assayed for VPg unlinkase activity. When [³⁵S]VPg-labeled virion RNA ([³⁵S]]VPg-PV RNA) isolated from purified poliovirus was incubated with GST-TDP2 or partially-purified VPg unlinkase, the unlinking of VPg was observed (FIG. 7a, top panel). Analysis of these reactions by 1% agarose gel electrophoresis verified that the release of VPg from vRNA was not due to RNA degradation (FIG. 7a, bottom panel). It was previously reported that VPg unlinkase can remove VPg from different picornavirus VPg-RNA substrates. Therefore, GST-TDP2 was incubated with [³⁵S]VPg-PV RNA or ³⁵S-methionine radiolabeled human rhinovirus 14 VPg linked to a poliovirus-rhinovirus chimeric RNA ([³⁵S]JVPg HRV-PV RNA). GST-TDP2, but not GST alone, was able to unlink VPg from either VPg-RNA substrate (FIG. 7b). The electrophoretic migration profiles of VPg generated by GST-TDP2 or partially purified VPg unlinkase were identical, but distinct from the slower migration of VPg-pUp, which is produced by the total degradation of vRNA using RNase A (compare lanes 2-5 to lane 6 and lanes 9-12 to lane 13 in FIG. 7b). These results demonstrate that TDP2 has authentic VPg unlinkase activity.

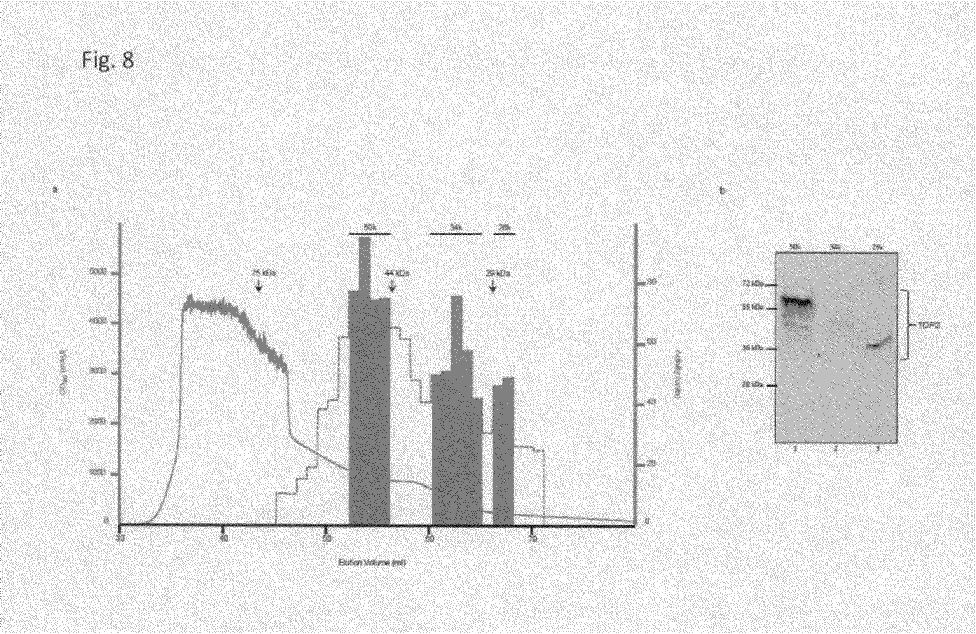

Although TDP2 is the only known 5′-tyrosyl-DNA phosphodiesterase found in vertebrate cells it was initially disregarded as a putative VPg unlinkase candidate for several reasons. (See, Zeng, Z., et al., I Biol. Chem. 286, 403-409 (2011), the disclosure of which is incorporated herein by reference.) First, it has been reported that VPg unlinkase cannot cleave the tyrosyl-nucleic acid linkage of a synthetic 5-tyrosyl-DNA substrate. (See, Shabanov, A. A., et al., Biokhimiia 61, 1106-1118 (1996), the disclosure of which is incorporated herein by reference.) However, in an effort to understand why VPg unlinkase is also unable to hydrolyze the serine-RNA linkage of the genome-linked protein of cowpea mosaic virus, it is necessary to consider the possibility that electrostatic interactions with tyrosine are important determinants for substrate recognition by VPg unlinkase, similar to the mechanisms used by apurinic/apyrimidinic endonuclease, cap-binding proteins and a wide range of other protein ligand interactions. (See, Drygin, Y. F., et al., FEBS Lett. 239, 343-346 (1988); De Varennes, A., et al., I Gen. Virol. 67, 2347 (1986); Gorman, M. A. et al. EMBO 1 16, 6548-6558 (1997); Fechter, P. and Brownlee, I Gen. Virol. 86, 1239-1249 (2005); and Zacharias, N. and Dougherty, D. A., Trends Pharmacol. Sci. 23, 28 1-287 (2002), the disclosures of which are incorporated herein by reference.) This model predicts that the 3,5-[¹²⁵ I]diiodotyrosine-labeled synthetic 5′-tyrosyl-DNA substrate used in the above-referenced work is incompatible with the active site of VPg unlinkase. Second, mass spectrometry analysis of fractions containing VPg unlinkase generated by previous purification protocols had not detected TDP2. (See, Rozovics, et al. (2011) cited above.) Considering that TDP2 is a fast and extremely low abundance enzyme it is likely that protein purity in relation to TDP2 abundance was an issue for these fractions. (See, Adhikari, S. et al., Anal. Biochem. 416, 112-116 (2011); and Pei, H. et al. Oncogene 22, 2699-2709 (2003), the disclosures of which are incorporated herein by reference.) Third, the molecular weight of full-length TDP2 did not correlate with any of the molecular weights reported for VPg unlinkase (−27 kDa and 24-30 kDa). (See, Gulevich, A. Y., et al., Biochemistry Mosc 66, 345-349 (2001), the disclosure of which is incorporated herein by reference.) Although this is true for the predominant forms of TDP2 described in the literature, at least three forms of TDP2 with apparent molecular masses ranging from 26 to 50 kDa have been detected. (FIG. 8b). All three forms of TDP2 co-eluted with the corresponding species of VPg unlinkase activity detected in crude extract (FIG. 8a). Because the phosphodiesterase domain of TDP2 is within the C-terminal portion of this protein, it can be surmised that previous studies may have only partially-purified a truncated form of TDP2. (See, Rodrigues-Lima, F., et al., Biochem. Biophys. Res. Commun. 285, 1274-1279 (2001), the disclosure of which is incorporated herein by reference.)

### Elucidation of Therapeutic Targets

A number of functional role(s) of TDP2 during a picornavirus infection have been proposed. (The potential disruptions of the viral replication cycle are depicted with dashed arrows in FIG. 1.) For example, although not to be bound by theory, it has been suggested that VPg unlinkase activity is involved in the maturation of picornavirus vRNA into mRNAs associated with translating polyribosomes. (See, Ambros, V., et al. (1978) & (1980); and Gulevich, et al., (2002), cited above.) An additional regulatory role for the unlinking of VPg by TDP2 may occur at the level of vRNA encapsidation. (See, Sangar, D. V., et al. (1981); Nomoto, et al., (1977); and Dorner, A., et al., FEBS Lett. 132, 219-223 (1981), the disclosures of each of which are incorporated herein by reference.) Since only VPg-linked RNA is encapsidated, TDP2 may be required to stimulate efficient viral RNA replication by inhibiting premature vRNA packaging. Because the levels of VPg unlinkase activity do not appear to change during poliovirus infection, this scenario suggests that TDP2 and viral proteins involved in vRNA packaging compete for nascent vRNAs. (Rozovics, et al., (2011), cited above.) Given the genetic and biochemical evidence that picornavirus RNA replication and encapsidation are coupled, TDP2 may be blocked from nascent vRNAs after sufficient levels of viral proteins have accumulated, resulting in increased production of progeny virions. (See, Nugent, C. I., et al., J. Virol. 73, 427-43 5 (1999); Liu, Y., et al., PLoS Pathog. 6, e1001066 (2010), the disclosures of which are incorporated herein by reference.) Therefore, picornavirus infections in cell culture carried out in the absence of TDP2 should result in a significant reduction in the levels of viral RNA replication (and ultimately, in virus yields) due to the premature packaging of vRNAs.

These functions make VPg unlinkase activity of TDP2 an attractive target for anti-viral therapeutics aimed at reducing the viral load in individuals infected with picornaviruses such as human rhinovirus or enterovirus 71 (EV7 I), especially given the morbidity in infants and children infected with these viruses. (See, Busse, W. W., Lemanske, R. F., Jr., and Gem, J. E. Lancet 376, 826-834 (2010); and Solomon, T., et al., Lancet Infect. Dis. 10, 778-790 (2010), the disclosures of which are incorporated herein by reference.)

In addition to these viral “therapeutic” targets, TDP2 is also implicated in DNA repair and cytoplasmic signaling pathways. Accordingly, it is possible that the suppression of TDP2 may allow for the therapeutic treatment some cancers. (See, e.g., Ledesma, F. C., et al., Nature, 461, 08444 (2009); Li, C., et al., Oncogene, 30, pp 3802-12 (2010); Zheng, z., et al., J. Biolo. Chem., 286:1, pp 403-9 (2011); Adhikari, S., et al., Anal. Biochem., 416, pp 112-16 (2011); and Li, C., et al., Cell Cycle, 10:19, pp 3274-83 (2011), the disclosure of which is incorporated herein by reference).

### Viral Therapeutics

In light of the above studies and results, some embodiments of the invention are directed to therapeutics targeted at suppressing the unlinkase activity of TDP2 in viral targets, and methods of treating viral infections using inhibitors of TDP2. The methods include identifying a subject having such a viral infection and administering a therapeutically effective amount of a specific inhibitor of TDP2, e.g., a therapeutic composition comprising the specific inhibitor of TDP2.

In some embodiments, the specific inhibitor of TDP2 is administered in a therapeutically effective amount as part of a course of treatment. As used in this context, to “treat” means to ameliorate at least one symptom of the viral disorder. A therapeutically effective amount can be an amount sufficient to prevent the onset of an acute episode or to shorten the duration of an acute episode, or to decrease the severity of one or more symptoms. In some embodiments, a therapeutically effective amount is an amount sufficient to reduce the viral load of the target virus in the patient.

Dosage, toxicity and therapeutic efficacy of the compounds can be determined, e.g., by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds that exhibit high therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the 1050 (i.e., the concentration of the test compound that achieves a half-maximal inhibition of symptoms) as determined in cell culture. Such information can be used to more accurately determine useful doses in humans. Levels in plasma may be measured, for example, by high performance liquid chromatography.

An “effective amount” is an amount sufficient to effect beneficial or desired results. For example, a therapeutic amount is one that achieves the desired therapeutic effect. This amount can be the same or different from a prophylactically effective amount, which is an amount necessary to prevent onset of disease or disease symptoms. An effective amount can be administered in one or more administrations, applications or dosages. A therapeutically effective amount of a composition depends on the composition selected. The compositions can be administered one from one or more times per day to one or more times per week; including once every other day. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of the compositions described herein can include a single treatment or a series of treatments.

In such embodiments, it will be understood that any suitable therapeutic target that suppresses such activity may be used with the current invention. For example, as discussed in the examples below, 5′FSBA has been tested and shown to suppress TDP2 activity. However, it is understood that conventional screening techniques may be used to find additional small molecule therapeutics from known libraries of such molecules that are readily and commercially available. As such, included in the current invention are methods for screening test compounds, e.g., polypeptides, polynucleotides, inorganic or organic large or small molecule test compounds, to identify agents useful in the suppression of TDP2 activity.

As used herein, “small molecules” refers to small organic or inorganic molecules of molecular weight below about 3,000 Daltons. The test compounds can be, e.g., natural products or members of a combinatorial chemistry library. A set of diverse molecules should be used to cover a variety of functions such as charge, aromaticity, hydrogen bonding, flexibility, size, length of side chain, hydrophobicity, and rigidity. Combinatorial techniques suitable for synthesizing small molecules are known in the art, e.g., as exemplified by Obrecht and Villalgordo, Solid-Supported Combinatorial and Parallel Synthesis of Small-Molecular-Weight Compound Libraries, Pergamon-Elsevier Science Limited (1998), and include those such as the “split and pool” or “parallel” synthesis techniques (the disclosure of which is incorporated herein by reference), solid-phase and solution-phase techniques. In addition, a number of small molecule libraries are commercially available. A number of suitable small molecule test compounds are listed in U.S. Pat. No. 6,503,713, for example, and are incorporated herein by reference.

Evaluation of these small molecules can be undertaken using any suitable technique, such as for example, application to a test sample, e.g., a cell or living tissue or organ, for evaluation. In a cultured or primary cell for example, the ability of the test compound to inhibit TDP2 may be evaluated. Methods for evaluating each target are well-known in the art. For example, ability to modulate expression of a protein can be evaluated at the gene or protein level, e.g., using quantitative PCR or immunoassay methods. In some embodiments, high throughput methods, e.g., protein or gene chips as are known in the art (see, e.g., Ch. 12, Genomics, in Griffiths et al., Eds. Modern genetic Analysis, 1999, W. H. Freeman and Company; Ekins and Chu, Trends in Biotechnology, 1999, 17:217-218; MacBeath and Schreiber, Science 2000, 289(5485):1760-1763; Simpson, Proteins and Proteomics: A Laboratory Manual, Cold Spring Harbor Laboratory Press; 2002; Hardiman, Microarrays Methods and Applications: Nuts & Bolts, DNA Press, 2003, the disclosures of each of which are incorporated herein by reference).

A test compound that has been screened by a method described herein and determined to inhibit TDP2, can be considered a candidate compound. A candidate compound that has been screened, e.g., in an in vivo model and determined to have a desirable effect on the disorder, e.g., on one or more symptoms of the disorder, can be considered a candidate therapeutic agent. Candidate therapeutic agents, once screened in a clinical setting, are therapeutic agents. Candidate compounds, candidate therapeutic agents, and therapeutic agents can be optionally optimized and/or derivatized, and formulated with physiologically acceptable excipients to form pharmaceutical compositions.

Test compounds identified as “hits” (e.g., test compounds that inhibit TDP2) in a first screen can be selected and systematically altered, e.g., using rational design, to optimize binding affinity, avidity, specificity, or other parameter. Such optimization can also be screened for using the methods described herein. Thus, in one embodiment, the invention includes screening a first library of compounds using a method known in the art and/or described herein, identifying one or more hits in that library, subjecting those hits to systematic structural alteration to create a second library of compounds structurally related to the hit, and screening the second library using the methods described herein.

Test compounds identified as hits can be considered candidate therapeutic compounds, useful in treating the target viral infection. A variety of techniques useful for determining the structures of “hits” can be used in the methods described herein, e.g., NMR, mass spectrometry, gas chromatography equipped with electron capture detectors, fluorescence and absorption spectroscopy. Thus, the invention also includes compounds identified as “hits” by the methods described herein, and methods for their administration and use in the treatment, prevention, or delay of development or progression of a disorder described herein.

Test compounds identified as candidate therapeutic compounds can be further screened by administration to an animal model of SPMS, as described herein. The animal can be monitored for a change in the disorder, e.g., for an improvement in a parameter of the disorder, e.g., a parameter related to clinical outcome.

### Therapeutic Targets

Although the above discussion has focused on picornaviruses it should be understood that any viral target that expresses a TDP2 unlinkase activity may be targeted by the therapeutics of the instant invention. Examples of other such viruses include, for example:
picornaviruses including: CVB3, EMCV, HRV16 and PVI;
plant viruses including: cowpea mosaic virus, sobemovirus, and rye grass mottle virus (see, De Varennes, et al., J. Gen. Vir., 67(11) pp. 2347 (1986); and Olspert, et al., FEBS Lett., 585(19) pp. 2979-85 (2011), the disclosures of which are incorporated herein by reference); and
caliciviruses, including: norwalk, feline calicivirus, and murine norovirus (see, Goodfellow, Curr. Opinion. Vir., (2011), the disclosure of which is incorporated herein by reference).

### Exemplary Embodiments

The invention is further described in the following examples, which do not limit the scope of the invention described in the claims.

### Methods and Materials

Except for an initial three hour methionine starvation during viral infection, generation of ³⁵S-methionine labeled vRNA was performed as previously described. (See, Rozovics, et al., (2011), cited above.) To generate the HeLa cell homogenate used for the isolation of VPg unlinkase, 5 ml of packed HeLa cells were homogenized by cryogenic grinding (Retsch) and then solubilized in PDEG1O buffer (20 mM phosphate buffer, pH 7.0, 5 mlvi DTT, 1 mM EDTA, 10% glycerol). The resulting extract was then subjected to the purification scheme described in the text. For the detection of VPg unlinkase activity, 2 μl aliquots of samples were incubated with 700 CPM ³⁵S-methionine labeled vRNA (−0.15 pmol) in a 20 jtl reaction volume containing PDEG1 0 buffer supplemented with 4 mM M2gCl at 30° C. for 3 minutes (enzyme detection) or 30 minutes (to detect nuclease activity). An additional I PV1 vRNA was included for visualization of reactions by 1% agarose electrophoresis. Reactions were either subjected to 13.5% Tris-tricine PAGE for two hours (for fast detection of activity) or 16 hours (to resolve VPg from VPg-pUp) and quantified as described previously. (Rozovics, et al., (2011), cited above.) Anti-TDP2 antibody was purchased from Santa Cruz Biotechnology (Anti-EAPII, sc-135214). The plasmid construct expressing GST-TDP2 was a developed by Dr. Runzhao Li, Emory University. (See, Pei, H. et al., Oncogene 22, 2699-2709 (2003), the disclosure of which is incorporated herein by reference.) Recombinant GST-TDP2 was expressed and purified on glutathione-agarose according to the manufacturer's protocol (GE Healthcare).

### Example 1

### Suppression of TDP2 by 5′FSBA

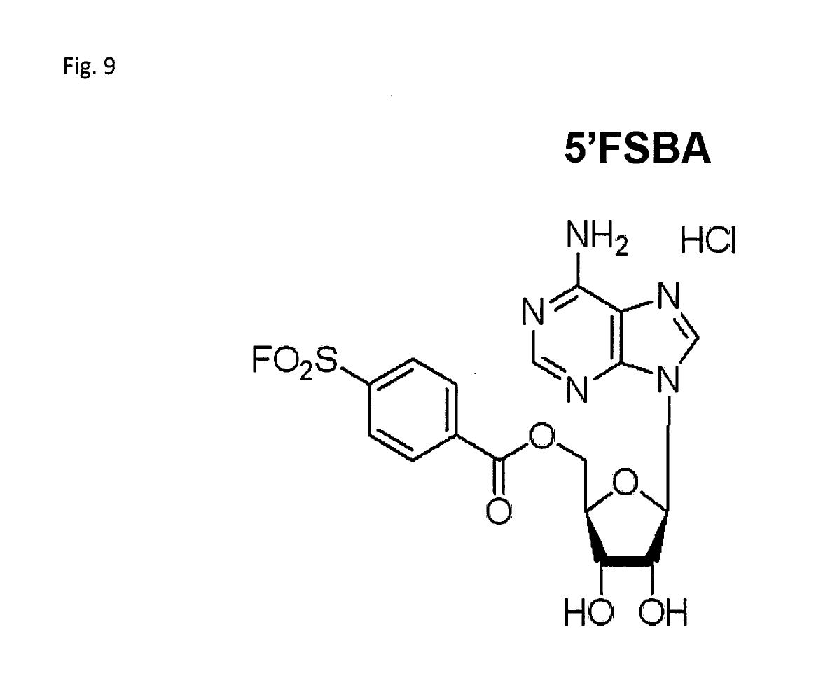

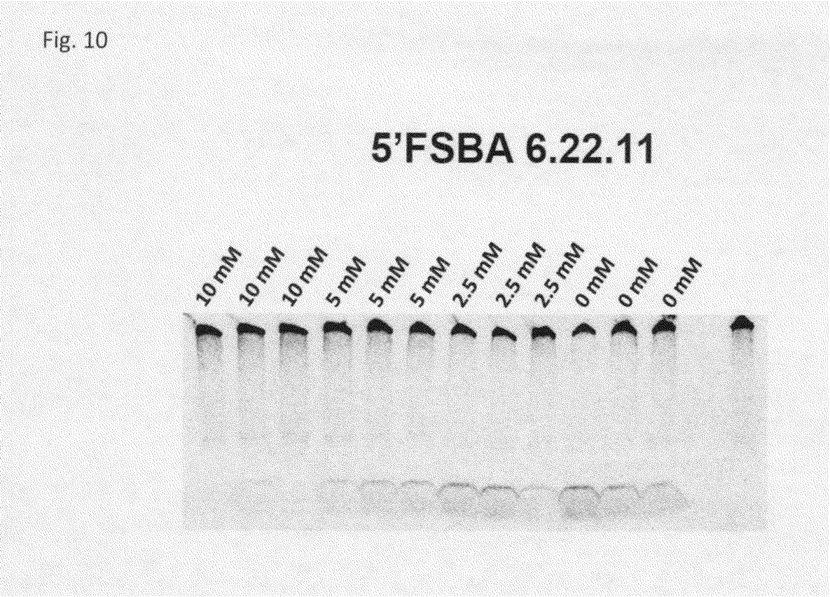

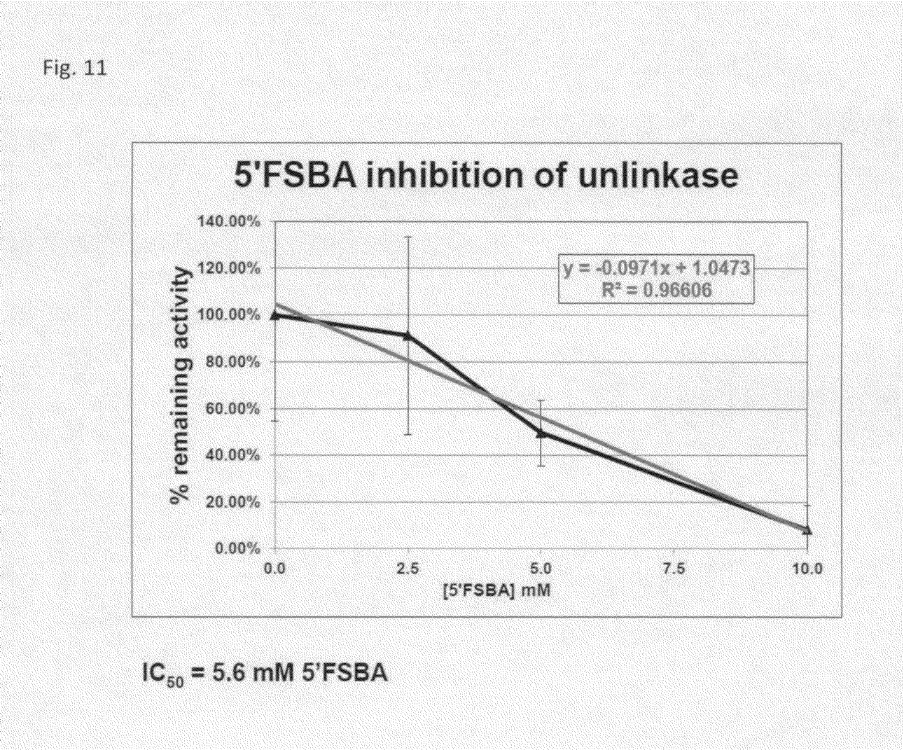

In one exemplary embodiment, the small molecule 5′FSBA (shown schematically in FIG. 9) was used to prove the efficacy of small molecule suppression of TDP2 activity. As shown in FIGS. 10 and 11, the introduction of the ‘5FSBA molecule effectively suppressed unlinkase activity by targeting the TDP2 enzyme.

### Example 2

### Effect of Suppression of TDP2 on Viral Replication

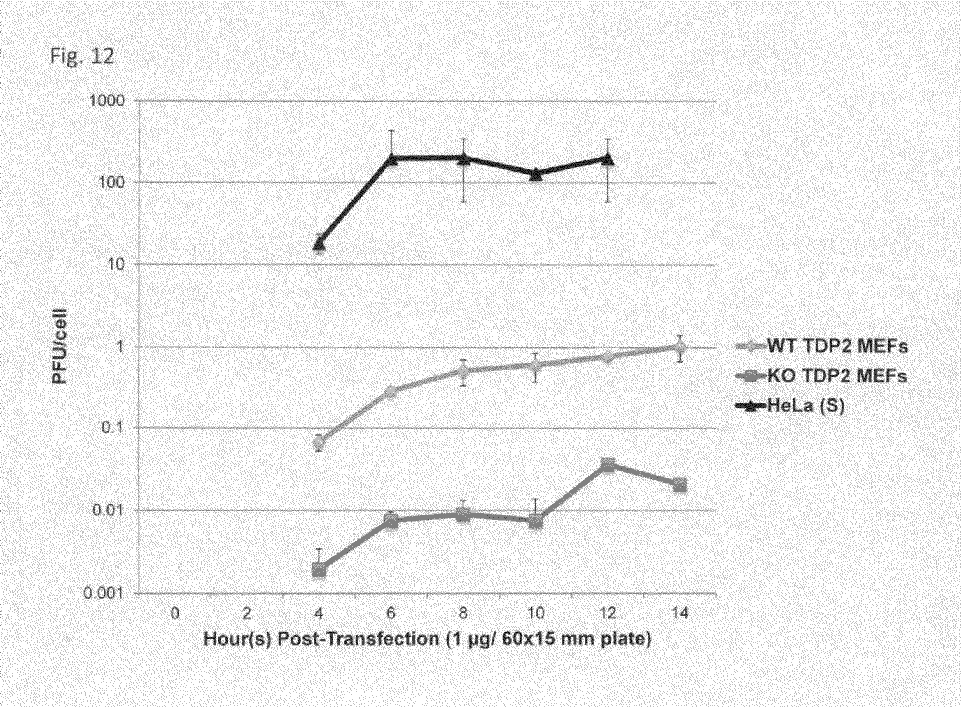

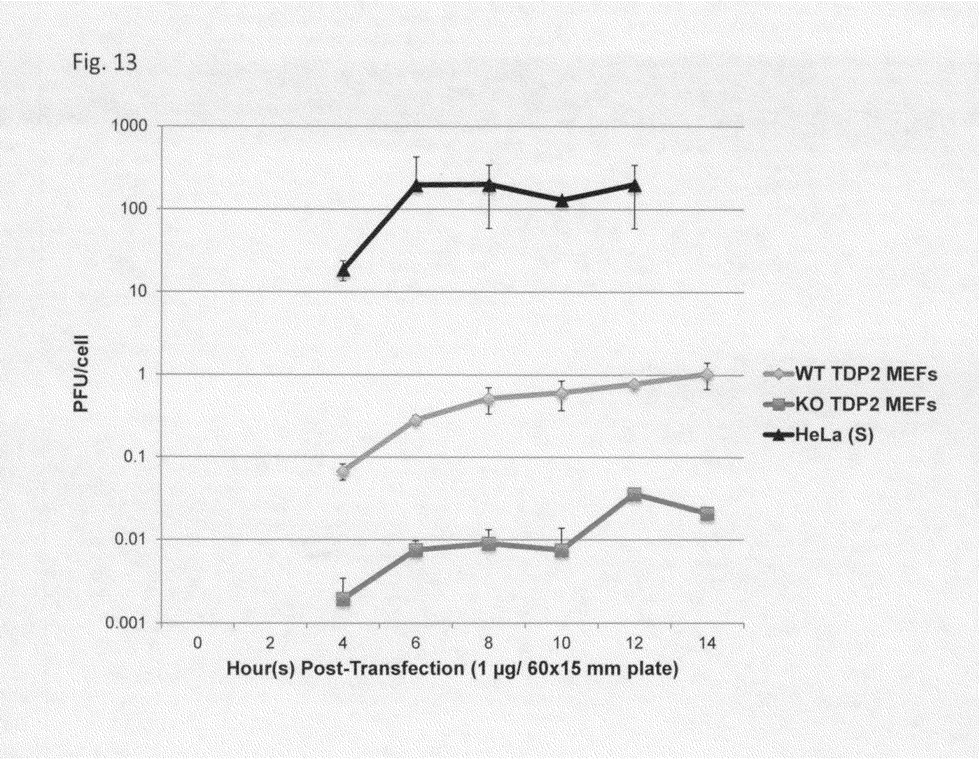

In another exemplary embodiment demonstrating the efficacy of the proposed therapeutic, a mouse embryo fibroblast (MEF) cell with a knockout of the TDP2 gene obtained from the University of Sussex was used to test the effect on viral replication in the poliovirus and the coxsackievirus. (See, e.g., Gomez-Herreros, F., et al., PLOS Genetics, 9:3, 1-16 (2013), the disclosure of which is incorporated herein by reference.) Because mouse cells lack the surface receptor for poliovirus (normally found on human cells), it was necessary to use polio virion RNA transfection to initiate the infection of the MEF(TDP2 KO) cells. For CVB3, this virus is capable of replication in mouse cells. As a result, the kinetics of virus replication after infection are much faster than those after virion RNA transfection. However, as shown in FIGS. 12 and 13 the effect is the same regardless of the mechanism used to initiate the infection for these two viruses. In particular, using these cells it is possible to show that poliovirus (PV) or coxsackievirus B3 (CVB3), both picornaviruses, replicate to 1.5-2.0 log 10 units lower in the absence of TDP2. These data support the proposal that TDP2 is important for picornavirus infections and that its suppression can be exploited as a novel target for antiviral therapeutics.

### SUMMARY

In summary, the embodiments describe the solution to a picornavirus mystery that has remained elusive for over three decades. It has now been discovered that unlinkase represents a novel target for anti-viral chemotherapy against a number of viruses including the broad class of picornaviruses and, as such, a potential pathway to ameliorate many diseases including the asthma exacerbations caused by human rhinovirus respiratory infections. Specifically, VPg unlinkase has now been identified as the DNA repair enzyme, 5′-tyrosyl-DNA phosphodiesterase-2 (TDP2). What is more, utilizing Western blot analysis, it has been possible to demonstrate the correlation of TDP2 expression levels with VPg unlinkase activity in different mammalian cell lines. Furthermore, it is shown that recombinant TDP2 has authentic VPg unlinkase activity and can release VPg from different picornavirus VPg-RNA substrates without impairing the integrity of viral RNA. In combination, these results reveal a novel RNA editing-like function for TDP2 and present a new role in host-pathogen interactions for this cellular enzyme, and represent a universal anti-viral therapeutic path against picornaviruses, and all other viruses with VPg unlinkase activity. What is more, TDP2 is the first enzyme to be ascribed a 5′-tyrosyl-RNA phosphodiesterase activity. The mammalian enzyme capable of 3′-tyrosyl-DNA phosphodiesterase activity (Tdp 1) harbors a limited 3′-nucleosidase activity capable of acting on both DNA and RNA substrates. (See, Interthal, H., et al., J. Biol. Chem. 280, 36518-36528 (2005), the disclosure of which is incorporated herein by reference.) However, to date this enzyme has not been reported to cleave tyrosyl-RNA linkages. Indeed, to the contrary it has been shown that a recombinant form of Tdpl does not possess VPg unlinkase activity, demonstrating that Tdpl is unable to cleave 5′-tyrosyl-RNA linkages.

In other words, during viral infection (of picornaviruses for example) the small viral protein VPg linked to the 5′ end of the virus genome is removed by a cellular enzymatic activity, termed “VPg unlinkase.” It has now been determined that since the removal of VPg specifically occurs concomitant with the change in the function of the viral genome, this activity has a regulatory role in the virus life cycle, and that TDP2 is the DNA repair enzyme responsible for this unlinkase activity. It has now further been discovered that targeting this unlinkase activity, and the activity of TDP2 more specifically, can function as a target for small molecule therapeutics. Accordingly, the current invention is directed to novel methods of treating viral infection by targeting therapies at this TDPs enzyme.

### DOCTRINE OF EQUIVALENTS

Those skilled in the art will appreciate that the foregoing examples and descriptions of various preferred embodiments of the present invention are merely illustrative of the invention as a whole, and that variations in the steps and various components of the present invention may be made within the spirit and scope of the invention. Accordingly, the present invention is not limited to the specific embodiments described herein but, rather, is defined by the scope of the appended claims.

What is claimed is:

1. An anti-viral therapeutic for viruses having VPg unlinkase activity, comprising:
a therapeutically effective amount of a VPg unlinkase enzyme suppressor.

2. The anti-viral therapeutic of claim 1, wherein the VPg unlinkase enzyme is TDP2.

3. The anti-viral therapeutic of claim 1, wherein the suppressor is 5′FSBA.

4. The anti-viral therapeutic of claim 1, wherein the virus is selected from the group consisting of picornaviruses, plant viruses and caliciviruses.

5. The anti-viral therapeutic of claim 4, wherein the picornavirus is selected from the group consisting of CVB3, EMCV, HRV16 VPg and PVI VPg.

6. The anti-viral therapeutic of claim 4, wherein the plant virus is selected from the group consisting of cowpea mosaic virus, sobemovirus, and rye grass mottle virus.

7. The anti-viral therapeutic of claim 4, wherein the calicivirus is selected from the group consisting of Norwalk virus, feline calicivirus, and murine norovirus.

8. A method of treating a viral infection for a virus having VPg unlinkase activity comprising:
administering a therapeutically effective amount of a VPg unlinkase enzyme suppressor.

9. The method of claim 8, wherein the VPg unlinkase enzyme is TDP2.

10. The method of claim 8, wherein the suppressor is 5′FSBA.

11. The method of claim 8, wherein the virus is selected form the group consisting of picornaviruses, plant viruses and caliciviruses.

12. The method of claim 11, wherein the picornvirus is selected from the group consisting of CVB3, EMCV, HRV16 VPg and PVI VPg.

13. The method of claim 11, wherein the plant virus is selected from the group consisting of cowpea mosaic virus, sobemovirus, and rye grass mottle virus.

14. The method of claim 11, wherein the calicivirus is selected from the group consisting of norwalk, feline calicivirus, and murine norovirus.

15. An assay for monitoring the release of VPg from genomic RNA generally comprising:
isolating a virion RNA containing an ³⁵S-labeled VPg;
producing a ³⁵S-VPg-nonanucleotide substrate;
entering the ³⁵S-VPg-nonanucleotide into a Tris-Tricine polyacrylamide gel; and
monitoring the cleavage of the labeled VPg from the substrate.

16. The assay of claim 15, wherein the step of isolating comprises:
labeling a virus-infected cell with a [³⁵S]methionine marker; and
purifying the labeled virion RNA from the virus-infected cells.

17. The assay of claim 16, wherein the purifying comprises sucrose gradient fractionation.

18. The assay of claim 15, wherein the producing comprises digesting the labeled VPg with RNAse T1.

19. A VPg unlinkase purification protocol comprising:
providing a VPg unlinkase containing target cell homogenate;
producing a supernatant form said target cell homogenate; and
sequentially fractionating the supernatant by heparin Sepharose, ssDNA-cellulose, anion exchange, size exclusion, and cation exchange chromatography to produce a substantially homogeneous enzyme preparation.

20. The purification protocol of claim 19, wherein the supernatant is produced by subjecting the target cell homogenate to high-speed centrifugation.
